(19) United States
(12) Patent Application Publication
(10) Pub. No.: US 2025/0367405 A1
(43) Pub. Date: Dec. 4, 2025

(54) TIP CONFIGURED TO BE DISPOSED AT DISTAL END PORTION OF MEDICAL DEVICE FOR INSERTION INTO LIVING BODY, AND MEDICAL TUBULAR BODY DELIVERY DEVICE

(71) Applicant: KANEKA CORPORATION, Osaka-shi, Osaka (JP)

(72) Inventors: Erina SANO, Settsu-shi (JP); Kentaro SUZUKI, Settsu-shi (JP); Kazuhiro SATO, Settsu-shi (JP)

(73) Assignee: KANEKA CORPORATION, Osaka-shi, Osaka (JP)

(21) Appl. No.: 18/836,616

(22) PCT Filed: Feb. 9, 2023
(86) PCT No.: PCT/JP2023/004331

§ 371 Date: Feb. 3, 2025

(30) Foreign Application Priority Data
Feb. 10, 2022 (JP) 2022-019800
Feb. 10, 2022 (JP) 2022-019801

(51) Int. Cl. A61M 25/00; A61L 29/04; A61L 29/18; A61M 25/01

(52) U.S. Cl. CPC A61M 25/0068; A61L 29/049; A61L 29/18; A61M 25/008; A61M 25/0108

(57) ABSTRACT

The purpose is to provide a tip configured to facilitate finding a position in a body cavity. A tip configured to be disposed at a distal end portion of a medical device for insertion into a living body includes a first region including a proximal end of the tip and a second region positioned distally to the first region, wherein the first region has higher total light transmittance measured with a D65 light source in comparison with the second region.

### TECHNICAL FIELD

The present invention relates to a tip configured to be disposed at a distal end portion of a medical device for insertion into a living body, and a medical tubular body delivery device.

### BACKGROUND ART

There have been conducted indwelling and the like of a stent at a stenosed site such as a stenosed digestive organ or a blood vessel with a stent delivery catheter, and there have been known various stent delivery catheters. Such stent delivery catheters each have a distal end often provided with a tip. For example, Patent Document 1 discloses a catheter tip located at a distal end of a catheter. The catheter tip includes a distal member made of a first material having a predetermined elastic modulus and constituting a distal end portion of the tip, and a proximal member made of a second material lower in elastic modulus than the first material, having a larger diameter in comparison with the distal member, constituting a proximal portion of the tip, and having a distal side provided with the distal member and a proximal side connected to the distal end of the catheter.

Patent Document 2 discloses a catheter tip including a first member connected to a distal end of a catheter, and a second member fixed to axially project from a distal end surface of the first member. The second member is made of a material harder than a material for the first member. At least one of the first member or the second member includes at least one anchor protrusion configured to be engaged with a remaining one of the first member and the second member.

### PRIOR ART DOCUMENTS

### Patent Documents

Patent Document 1: JP-A-2014-195556
Patent Document 2: WO 2018/181962 A

### SUMMARY OF THE INVENTION

### Problems to be Solved by the Invention

When a medical tubular body such as a stent is indwelt in a stenosed portion such as a bile duct with a stent delivery catheter, the stent may have conventionally been disposed in the stenosed portion with an endoscope or the like. However, the tip can be hardly checked in terms of a position in a body cavity with the conventional stent delivery catheter. Any other catheter tip is required to have improvement in visual recognition. A first aspect of the present invention has been achieved in view of the above problem, and it is an object of the first aspect of the present invention to provide a tip configured to facilitate finding a position in a body cavity. It is another object to provide a medical tubular body delivery device including the tip configured to facilitate finding the position in the body cavity.

When a medical tubular body such as a stent is conveyed to a stenosed portion like a digestive organ or a blood vessel with a stent delivery catheter, it has conventionally been necessary to accurately find a distal end position of the stent delivery catheter in a living body at some stenosed site. When a different catheter is adopted, it has also been necessary to accurately find a distal end position of the catheter in some treatment target portion. Development of a tip has thus recently been required for easily finding a distal end position of a catheter in a living body. A second aspect of the present invention has been achieved in view of the above problem, and it is an object of the second aspect of the present invention to provide a tip configured to facilitate finding a position in a living body. It is another object to provide a medical tubular body delivery device including the tip configured to facilitate finding the position in the living body.

### Solutions to the Problems

According to the first aspect of the present invention, there is provided a tip configured to be disposed at a distal end portion of a medical device for insertion into a living body in order to solve the above problem. The tip is configured as follows.
[1] A tip configured to be disposed at a distal end portion of a medical device for insertion into a living body, the tip including:
a first region including a proximal end of the tip; and
a second region positioned distally to the first region; wherein
the first region has higher total light transmittance measured with a D65 light source in comparison with the second region.

When the first region has higher total light transmittance in comparison with the second region as described above, in other words, when the second region is lower in total light transmittance than the first region, the second region is decreased in transparency for higher visibility of the tip. This facilitates finding a position of the tip in a body cavity. Furthermore, the first region is improved in transparency when the first region is higher in total light transmittance than the second region. When the tip thus configured is applied as a tip of a medical tubular body delivery device, a medical tubular body provided near the proximal end of the tip can be easily checked in terms of a position of a distal end. This facilitates placement of the medical tubular body in a stenosed portion.

The tip according to the first aspect of the present invention is preferably configured in a manner described in any one of the following [2] to [15].
[2] The tip according to [1], wherein the first region has less haze measured with the D65 light source in comparison with the second region.
[3] The tip according to [1] or [2], wherein
the second region includes a colorant, and
the first region does not include any colorant or includes a colorant smaller in mass percent content than the colorant in the second region.
[4] The tip according to [3], wherein
the colorant is a pigment, a dye, or a mixture of the pigment and the dye.
[5] The tip according to [3] or [4], wherein
the first region includes a lumen extending from a proximal end to a distal end, and
the second region includes a lumen extending from a proximal end to a distal end and communicating with the lumen in the first region.
[6] The tip according to [5], wherein at least part of an inner surface of the lumen in the second region includes the colorant.
[7] The tip according to any one of [3] to [6], wherein at least part of an outer surface of the second region includes the colorant.
[8] The tip according to any one of [3] to [7], wherein the tip has a distal end including the colorant.
[9] The tip according to any one of [1] to [8], wherein
the second region includes a crystalline resin, and
the first region does not include any crystalline resin or includes a crystalline resin smaller in mass percent content than the crystalline resin in the second region.
[10] The tip according to any one of [1] to [9], wherein
the second region includes a crystalline resin, and
the first region does not include any crystalline resin or includes a crystalline resin lower in crystallinity than the crystalline resin in the second region.
[11] The tip according to any one of [1] to [10], wherein the first region includes an amorphous resin.
[12] The tip according to any one of [1] to [11], the tip
including at least one diameter increasing portion having an outer diameter increasing toward a distal end of the tip, including at least one diameter decreasing portion having an outer diameter decreasing toward the distal end of the tip, or including the at least one diameter increasing portion and the at least one diameter decreasing portion; wherein
a most proximal diameter decreasing portion most proximally positioned in the at least one diameter decreasing portion is positioned distally to a most distal diameter increasing portion most distally positioned in the at least one diameter increasing portion.
[13] The tip according to [12], wherein the most distal diameter increasing portion is smaller in slant angle from an axial direction of the tip than the most proximal diameter decreasing portion.
[14] The tip according to [12] or [13], wherein the first region has a distal end positioned distally to a distal end of the most distal diameter increasing portion.
[15] The tip according to any one of [12] to [14], wherein the first region has a distal end positioned proximally to a proximal end of the most proximal diameter decreasing portion.

According to the first aspect of the present invention, there is provided a medical tubular body delivery device in order to solve the above problem. The medical tubular body delivery device is configured as follows.
[16] A medical tubular body delivery device configured to deliver a medical tubular body into a living body, the medical tubular body delivery device including:
an inner tube;
an outer tube including a lumen, at least part of the inner tube being disposed in the lumen;
the tip according to any one of [1] to [15], the tip being fixed to at least part of the distal end portion of the inner tube; and
a medical tubular body disposed between an outer surface of the distal end portion of the inner tube and an inner surface of the outer tube.

Furthermore, the medical tubular body delivery device according to the first aspect of the present invention is preferably configured in a manner described in any one of [17] and [18].
[17] The medical tubular body delivery device according to [16], wherein the medical tubular body has a distal end positioned proximally to a distal end of the first region of the tip.
[18] The medical tubular body delivery device according to [16] or [17], wherein the medical tubular body has a distal end positioned distally to a proximal end of the first region of the tip.

According to the second aspect of the present invention, there is provided a tip configured to be disposed at a distal end portion of a medical device for insertion into a living body in order to solve the above problem. The tip is configured as follows.
[19] A tip configured to be disposed at a distal end portion of a medical device for insertion into a living body, the tip including:
a first region including a proximal end of the tip; and
a second region positioned distally to the first region and including a resin and a radiopaque filler; wherein
the first region includes a resin and does not include any radiopaque filler, or includes a resin and a radiopaque filler smaller in mass percent content than the radiopaque filler in the second region.

As described above, the tip including the radiopaque filler facilitates finding a position of the tip during X-ray fluoroscopy. Furthermore, the first region of the tip not including any radiopaque filler or including a radiopaque filler smaller in content than the radiopaque filler in the second region achieves relatively improved radiotransparency at the proximal end portion of the tip. Applying the tip thus configured as a tip of a medical tubular body delivery device facilitates accurately finding a position of a distal end or the like of a radiopaque medical tubular body positioned near the proximal end of the tip during X-ray fluoroscopy. The radiopaque filler adopted as a radiopaque substance in the tip is less likely to cause large unevenness in a lumen of the tip in comparison with a case where a radiopaque marker band is provided around the lumen of the tip. This facilitates avoidance of damage to the lumen of the tip due to insertion, removal, or the like of a guide wire.

The tip according to the second aspect of the present invention is preferably configured in a manner described in any one of the following [20] to [28].
[20] The tip according to [19], wherein the first region includes the resin and does not include any radiopaque substance.
[21] The tip according to [19] or [20], wherein
the first region includes a lumen extending from a proximal end to a distal end, and
the second region includes a lumen extending from a proximal end to a distal end and communicating with the lumen in the first region.
[22] The tip according to [21], wherein at least part of an inner surface of the lumen in the second region includes the radiopaque filler.
[23] The tip according to any one of [19] to [22], wherein at least part of an outer surface of the second region includes the radiopaque filler.
[24] The tip according to any one of [19] to [23], wherein the tip has a distal end including the radiopaque filler.
[25] The tip according to any one of [19] to [24], the tip
including at least one diameter increasing portion having an outer diameter increasing toward a distal end of the tip, including at least one diameter decreasing portion having an outer diameter decreasing toward the distal end of the tip, or including the at least one diameter increasing portion and the at least one diameter decreasing portion; wherein
a most proximal diameter decreasing portion most proximally positioned in the at least one diameter decreasing portion is positioned distally to a most distal diameter increasing portion most distally positioned in the at least one diameter increasing portion.
[26] The tip according to [25], wherein the most distal diameter increasing portion is smaller in slant angle from an axial direction of the tip than the most proximal diameter decreasing portion.
[27] The tip according to [25] or [26], wherein the first region has a distal end positioned distally to a distal end of the most distal diameter increasing portion.
[28] The tip according to any one of [25] to [27], wherein the first region has a distal end positioned proximally to a proximal end of the most proximal diameter decreasing portion.

According to the second aspect of the present invention, there is provided a medical tubular body delivery device in order to solve the above problem. The medical tubular body delivery device is configured as follows.
[29] A medical tubular body delivery device configured to deliver a medical tubular body into a living body, the medical tubular body delivery device including:
an inner tube;
an outer tube including a lumen, at least part of the inner tube being disposed in the lumen;
the tip according to any one of [19] to [28], the tip being fixed to at least part of a distal end portion of the inner tube; and
a medical tubular body disposed between an outer surface of the distal end portion of the inner tube and an inner surface of the outer tube, and including a radiopaque substance.

Furthermore, the medical tubular body delivery device according to the second aspect of the present invention is preferably configured in a manner described in any one of [30] to [35].
[30] The medical tubular body delivery device according to [29], wherein the medical tubular body has a distal end positioned proximally to a distal end of the first region of the tip.
[31] The medical tubular body delivery device according to [29] or [30], wherein the medical tubular body has a distal end positioned distally to a proximal end of the first region of the tip.
[32] The medical tubular body delivery device according to any one of [29] to [31], wherein the outer tube has a distal end portion including a radiopaque region having radiopacity.
[33] The medical tubular body delivery device according to [32], wherein the radiopaque region is positioned proximally to a distal end of the first region of the tip.
[34] The medical tubular body delivery device according to [32] or [33], wherein the radiopaque region is positioned distally to a proximal end of the first region of the tip.
[35] The medical tubular body delivery device according to any one of [32] to [34], wherein the radiopaque region includes a radiopaque marker, includes a resin and a radiopaque filler, or includes the radiopaque marker, the resin, and the radiopaque filler.

As described in any one of the following [36] to [52], any of respective configurations according to the first aspect of the present invention may be combined with any of respective configurations according to the second aspect of the present invention.
[36] A tip according to any one of [1] to [15], wherein
the second region includes a resin and a radiopaque filler, and
the first region includes a resin and does not include any radiopaque filler, or includes a resin and a radiopaque filler smaller in mass percent content than the radiopaque filler in the second region.
[37] The tip according to [36], wherein the first region includes the resin and does not include any radiopaque substance.
[38] The tip according to [36] or [37], wherein
the first region includes a lumen extending from a proximal end to a distal end, and
the second region includes a lumen extending from a proximal end to a distal end and communicating with the lumen in the first region.
[39] The tip according to [38], wherein at least part of an inner surface of the lumen in the second region includes the radiopaque filler.
[40] The tip according to any one of [36] to [39], wherein at least part of an outer surface of the second region includes the radiopaque filler.
[41] The tip according to any one of [36] to [40], wherein the tip has a distal end including the radiopaque filler.
[42] The tip according to any one of [36] to [41], the tip
including at least one diameter increasing portion having an outer diameter increasing toward a distal end of the tip, including at least one diameter decreasing portion having an outer diameter decreasing toward the distal end of the tip, or including the at least one diameter increasing portion and the at least one diameter decreasing portion; wherein
a most proximal diameter decreasing portion most proximally positioned in the at least one diameter decreasing portion is positioned distally to a most distal diameter increasing portion most distally positioned in the at least one diameter increasing portion.
[43] The tip according to [42], wherein the most distal diameter increasing portion is smaller in slant angle from an axial direction of the tip than the most proximal diameter decreasing portion.
[44] The tip according to [42] or [43], wherein the first region has a distal end positioned distally to a distal end of the most distal diameter increasing portion.
[45] The tip according to any one of [42] to [44], wherein the first region has a distal end positioned proximally to a proximal end of the most proximal diameter decreasing portion.
[46] A medical tubular body delivery device configured to deliver a medical tubular body into a living body, the medical tubular body delivery device including:
an inner tube;
an outer tube including a lumen, at least part of the inner tube being disposed in the lumen;
the tip according to any one of [36] to [45], the tip being fixed to at least part of a distal end portion of the inner tube; and
a medical tubular body disposed between an outer surface of the distal end portion of the inner tube and an inner surface of the outer tube, and including a radiopaque substance.
[47] The medical tubular body delivery device according to [46], wherein the medical tubular body has a distal end positioned proximally to a distal end of the first region of the tip.
[48] The medical tubular body delivery device according to [46] or [47], wherein the medical tubular body has a distal end positioned distally to a proximal end of the first region of the tip.
[49] The medical tubular body delivery device according to any one of [46] to [48], wherein the outer tube has a distal end portion including a radiopaque region having radiopacity.
[50] The medical tubular body delivery device according to [49], wherein the radiopaque region is positioned proximally to a distal end of the first region of the tip.
[51] The medical tubular body delivery device according to [49] or [50], wherein the radiopaque region is positioned distally to a proximal end of the first region of the tip.
[52] The medical tubular body delivery device according to any one of [49] to [51], wherein the radiopaque region includes a radiopaque marker, includes a resin and a radiopaque filler, or includes the radiopaque marker, the resin, and the radiopaque filler.

### Effects of the Invention

The above configurations according to the first aspect of the present invention achieve provision of the tip that facilitates finding a position in a body cavity. The above configurations also achieve provision of the medical tubular body delivery device including the tip that facilitates finding the position in the body cavity.

The above configurations according to the second aspect of the present invention achieve provision of the tip that facilitates finding a position in a body cavity. The above configurations also achieve provision of the medical tubular body delivery device including the tip that facilitates finding the position in the body cavity.

### BRIEF DESCRIPTION OF THE DRAWINGS

FIG. 1 is a side view a medical tubular body delivery device according to a first embodiment of the invention.

FIG. 2 is a sectional view taken in an axial direction indicated in FIG. 1.

FIG. 3 is a sectional view of a tip depicted in FIG. 2 according to a modification example.

FIG. 4 is a sectional view of the tip depicted in FIG. 2 according to another modification example.

FIG. 5 is a sectional view of the tip depicted in FIG. 2 according to still another modification example.

FIG. 6 is a sectional view of the tip depicted in FIG. 2 according to a different modification example.

FIG. 7 is a sectional view of the tip depicted in FIG. 2 according to another different modification example.

FIG. 8 is a sectional view of the tip depicted in FIG. 2 according to still another different modification example.

FIG. 9 is a sectional view taken in an axial direction of a medical tubular body delivery device according to another embodiment.

FIG. 10 is a sectional view of the tip and an outer tube depicted in FIG. 2 according to a modification example.

FIG. 11 is a side view of a medical tubular body delivery device according to a second embodiment of the invention.

FIG. 12 is a sectional view taken in an axial direction indicated in FIG. 11.

FIG. 13 is a schematic view exemplifying a tip and a medical tubular body during X-ray fluoroscopy.

FIG. 14 is a schematic view of a tip and a medical tubular body according to the embodiment during X-ray fluoroscopy.

FIG. 15 is a sectional view of a tip depicted in FIG. 12 according to a modification example.

FIG. 16 is a sectional view of the tip depicted in FIG. 12 according to another modification example.

FIG. 17 is a sectional view of the tip depicted in FIG. 12 according to still another modification example.

FIG. 18 is a sectional view of the tip depicted in FIG. 12 according to a different modification example.

FIG. 19 is a sectional view of the tip depicted in FIG. 12 according to another different modification example.

FIG. 20 is a sectional view of the tip depicted in FIG. 12 according to still another different modification example.

FIG. 21 is a sectional view taken in an axial direction of a medical tubular body delivery device according to still another embodiment.

FIG. 22 is a sectional view of the tip and an outer tube depicted in FIG. 12 according to a modification example.

### MODE FOR CARRYING OUT THE INVENTION

More specific description will now be made to the following embodiments of the present invention. The present invention should not be limited by the following embodiments and can obviously be implemented with appropriate changes within a range applicable to purport of the above and below description. Such changes will be all included in the technical scope of the present invention. Some of the drawings may not include reference signs of members for convenience, in which case the specification or any other appropriate drawing should be referred to. Various members in the drawings may not be depicted in actual sizes, because contribution to comprehension of the features of the present invention is prioritized in the drawings.

According to a first embodiment of the present invention, a tip is configured to be disposed at a distal end portion of a medical device for insertion into a living body. The tip includes a first region including a proximal end of the tip, and a second region positioned distally to the first region. The first region bas higher total light transmittance measured with a D65 light source in comparison with the second region.

When the first region has higher total light transmittance in comparison with the second region as described above, in other words, when the second region is lower in total light transmittance than the first region, the second region is decreased in transparency for higher visibility of the tip. This facilitates finding a position of the tip in a body cavity. Furthermore, the first region is improved in transparency when the first region is higher in total light transmittance than the second region. When the tip thus configured is applied as a tip of a medical tubular body delivery device, a medical tubular body provided near the proximal end of the tip can be easily checked in terms of a position of a distal end.

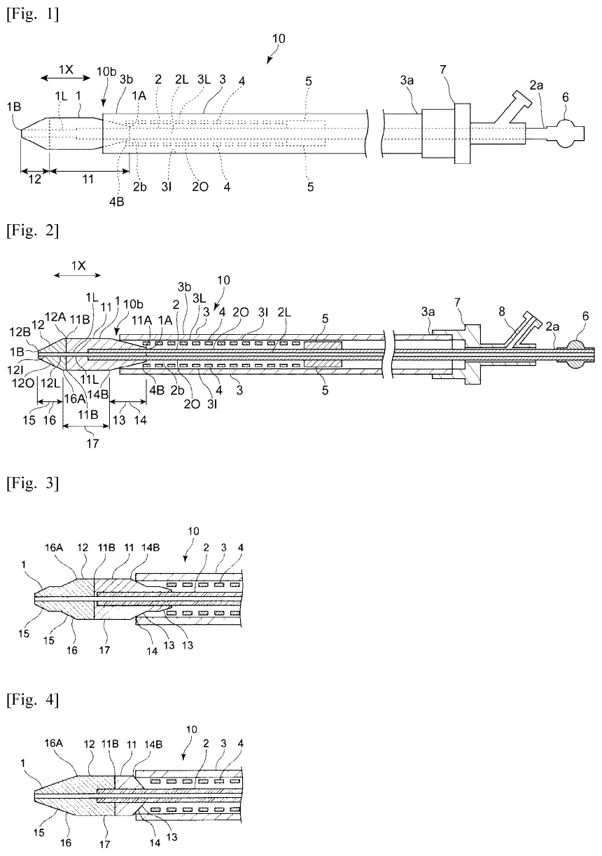

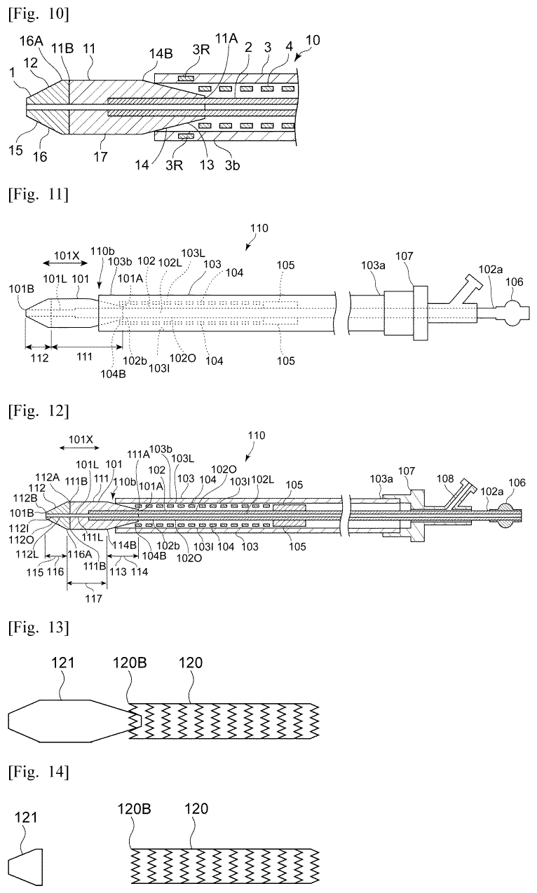

With reference to FIGS. 1 to 10, the following description initially refers to the tip according to the embodiment, the tip being configured to be disposed at a distal end portion of a medical device for insertion into a living body, and then refers to a medical tubular body delivery device according to the embodiment. FIG. 1 is a side view of the medical tubular body delivery device according to the embodiment. FIG. 2 is a sectional view taken in an axial direction indicated in FIG. 1. Each of FIGS. 3 to 8 is a sectional view of a tip depicted in FIG. 2 according to a modification example. FIG. 9 is a sectional view taken in an axial direction of a medical tubular body delivery device according to another embodiment. FIG. 10 is a sectional view of the tip and an outer tube depicted in FIG. 2 according to a modification example.

FIGS. 1 and 2 depict a tip 1 according to the embodiment, and the tip 1 is configured to be disposed at a distal end portion of a medical device for insertion into a living body. Specifically, the tip 1 is preferred to be disposed at a distal end portion 10b of a medical tubular body delivery device 10 configured to deliver a medical tubular body 4 into a living body. The tip 1 includes a first region 11 including a proximal end IA of the tip 1, and a second region 12 positioned distally to the first region 11. The first region 11 has higher total light transmittance measured with the D65 light source in comparison with the second region 12.

When the first region 11 has higher total light transmittance in comparison with the second region 12 as described above, in other words, when the second region 12 is lower in total light transmittance than the first region 11, the second region 12 is decreased in transparency for higher visibility of a distal end portion of the tip 1. This facilitates finding a position of the tip 1 in a body cavity.

Furthermore, the first region 11 is improved in transparency when the first region 11 is higher in total light transmittance than the second region 12 as described above. When the tip 1 thus configured is applied as a tip of the medical tubular body delivery device 10, the medical tubular body 4 provided near the proximal end 1A of the tip 1 can be easily checked in terms of a position of a distal end 4B. This facilitates placement of the medical tubular body 4 in a stenosed portion. Furthermore, the first region 11 having high transparency enables checking whether or not the distal end 4B and the like of the medical tubular body 4 are damaged immediately before the medical tubular body 4 is disposed in the stenosed portion.

The first region 11 has total light transmittance (%) that is preferably 1.01 times or more total light transmittance (%) of the second region 12, more preferably 1.05 times or more, further preferably 1.2 times or more, still further preferably 1.5 times or more, and particularly preferably 2.0 times or more. This facilitates exhibition of the effects achieved by the first region 11 and the second region 12. Meanwhile, such a magnification may be exemplarily 5.0 times or less.

The first region 11 is preferred to have less haze measured with the D65 light source in comparison with the second region 12. Less haze leads to improved transparency whereas more haze leads to lowered transparency. Accordingly, this configuration facilitates exhibition of the effects achieved by the first region 11 and the second region 12. The first region 11 has haze (%) that is preferably 0.5 times or less haze (%) of the second region 12, more preferably 0.2 times or less, further preferably 0.1 times or less, still further preferably 0.04 times or less, and particularly preferably 0.01 times or less. Meanwhile, such a magnification may be exemplarily 0.0001 times or more.

Total light transmittance can be found by substantially radially cutting each region of the tip 1 and measuring a cut piece thus obtained in accordance with the method prescribed in JIS K 7361-1:1997. Haze can be found by similarly measuring the cut piece in accordance with the method prescribed in JIS K 7136:2000.

Preferably, the second region 12 includes a colorant, and the first region 11 does not include any colorant or includes a colorant smaller in mass percent content than the colorant in the second region 12. This achieves lowered transparency in the second region 12 as well as relatively high transparency in the first region 11. Accordingly, the first region 11 more preferably includes no colorant. The second region 12 can have lowered transparency also by including a radiopaque filler. Accordingly, it is preferred that the second region 12 includes the radiopaque filler and the first region 11 does not include any radiopaque filler or includes a radiopaque filler smaller in mass percent content than the radiopaque filler in the second region 12. Furthermore, the first region 11 more preferably includes no radiopaque substance. Description of a tip according to a second embodiment of the present invention to be provided later may be referred to for details of the radiopaque filler and the radiopaque substance.

The colorant is preferably a pigment, a dye, or a mixture of the pigment and the dye, and is more preferably the pigment. The pigment is preferably an organic pigment, an inorganic pigment, or a mixture of the organic pigment and the inorganic pigment. Examples of the organic pigment include an anthraquinone-based pigment, a phthalocyanine pigment, a quinacridone-based pigment, a perylene-based pigment, an azo pigment such as an azo lake-based pigment or a condensed azo-based pigment, a benzimidazolone based pigment, a thioindigo pigment, an isoindolinon pigment, and a quinophthalone pigment. Examples of the inorganic pigment include carbon black, an iron oxide, a titanium oxide, chrome yellow, zinc yellow, ultramarine blue, prussian blue, and a red iron oxide. The dye is preferably an anthraquinone-based dye, a perylene-based pigment, a perinone-based dye, a solvent dye, or a mixture of any of these dyes. The colorant is preferably a blue colorant, a green colorant, a yellow colorant, a black colorant, or a mixture of any of these colorants.

In a case where the first region 11 includes a colorant, the colorant in the first region 11 has a mass percent content that is preferably 0.8 times or less a mass percent content of the colorant in the second region 12, more preferably 0.5 times or less, further preferably 0.2 times or less, and still further preferably 0.1 times or less. Meanwhile, such a magnification may be 0.01 times or more. The first region 11 including a colorant is preferred to include a kneaded product containing a resin and the colorant. Measurement of a content of a colorant in each region can be achieved by substantially radially cutting each region and obtaining a mass ratio (mass percent) of the colorant to 100 mass percent of a cut piece.

The first region 11 preferably includes at least one uncolored tubular layer containing a resin and not containing any colorant, and preferably consists of the at least one uncolored tubular layer. The first region 11 including a colorant is preferred to include the colorant at an entirely even concentration.

Preferably, the second region 12 includes a crystalline resin, and the first region 11 does not include any crystalline resin or includes a crystalline resin smaller in mass percent content than the crystalline resin in the second region 12. A crystalline resin leads to lowered transparency. This configuration can thus achieve lowered transparency in the second region 12 as well as relatively high transparency in the first region 11.

In a case where the first region 11 includes a crystalline resin, the crystalline resin in the first region 11 bas a mass percent content that is preferably 0.8 times or less a mass percent content of the crystalline resin in the second region 12, more preferably 0.5 times or less, further preferably 0.2 times or less, and still further preferably 0.1 times or less. Meanwhile, such a magnification may be 0.01 times or more. Measurement of a content of a crystalline resin or the like in each region can be achieved by substantially radially cutting each region and obtaining a mass ratio (mass percent) of the crystalline resin or the like to 100 mass percent of a cut piece.

Preferably, the second region 12 includes a crystalline resin, and the first region 11 does not include any crystalline resin or includes a crystalline resin lower in crystallinity than the crystalline resin in the second region 12. A resin having high crystallinity leads to lowered transparency, whereas a resin having low crystallinity leads to improved transparency. This configuration can thus achieve lowered transparency in the second region 12 as well as improved transparency in the first region 11.

The first region 11 may include a crystalline resin, may include an amorphous resin, or may include the crystalline resin and the amorphous resin. The first region 11 is preferred to include the crystalline resin and the amorphous resin or include the amorphous resin. The first region 11 is more preferred to include the amorphous resin. This achieves improved transparency in the first region 11.

The crystalline resin mentioned above exhibits a clear endothermic peak in differential scanning calorimetry (DSC). The amorphous resin mentioned above does not exhibit a clear endothermic peak in DSC. A clear endothermic peak corresponds to an endothermic peak having a half width within 15° C. in DSC. Examples of a temperature increase rate in DSC include 10° C. per minute.

The crystallinity mentioned above may be obtained through DSC or the like. For example, the crystallinity can be calculated by obtaining fusion heat quantity of a sample in accordance with DSC and dividing the fusion heat quantity by perfect crystal fusion heat quantity corresponding to a material for the sample.

As described above, the second region 12 is lower in total light transmittance than the first region 11. This leads to lowered transparency in the second region 12 for higher visibility of the tip 1. This facilitates finding a position of the tip 1 in a body cavity. The second region 12 may be positioned proximally to a distal end 1B of the tip 1, but preferably includes the distal end 1B of the tip 1. This achieves higher visibility of the distal end 1B of the tip 1 and facilitates insertion of the tip 1 into a stenosed portion. Some operation methods may include inserting a guide wire from the distal end 1B of the tip 1. The second region 12 including the distal end 1B of the tip 1 achieves higher visibility of the distal end 1B of the tip 1 and facilitates insertion of the guide wire from the distal end 1B.

The second region 12 is preferably disposed adjacent to the first region 11. This leads to more obvious opaqueness of the second region 12 for higher visibility of the tip 1.

The second region 12 preferably includes a kneaded product containing a resin and a colorant, more preferably includes a kneaded product containing a crystalline resin and a colorant, and further preferably consists of the kneaded product. This achieves lowered transparency in the second region 12.

At least part of an inner surface 12I around a lumen 12L in the second region 12 preferably includes the colorant. This achieves lowered transparency at and around a central axis of the second region 12. More preferably, the inner surface 12I of the second region 12 entirely includes the colorant.

Preferably, at least part of an outer surface 12O in the second region 12 includes the colorant. This achieves lowered transparency at and near an outline of the second region 12. More preferably, the outer surface 12O in the second region 12 entirely includes the colorant.

The distal end 1B of the tip 1 preferably includes a colorant. This facilitates finding a position of the distal end 1B of the tip 1. This also facilitates delicate operation such as passing the tip 1 through a mesh of a stent already conveyed into a body cavity and developed.

The second region 12 preferably includes at least one colored tubular layer containing a resin and a colorant, and more preferably consists of the at least one colored tubular layer. The second region 12 is preferred to include the colorant at an entirely even concentration.

Preferred examples of a resin provided in the first region 11 or the second region 12 include polyethylene, polypropylene, polyamide, polyurethane, polyester, silicone, polycarbonate, polyether sulfone, a polyamide-based elastomer, a fluororesin such as PTFE or PFA, an ABS resin, and a mixture of any of these resins. The resin included in the first region 11 and the resin included the second region 12 may be different from each other, but are preferably the same. The first region 11 and the second region 12 may include an additive such as a hydrolysis inhibitor, a flame retarder, a filler, or a crosslinker.

The tip 1 is preferred to include at least one diameter increasing portion 13 having an outer diameter increasing toward the distal end 1B of the tip 1, include at least one diameter decreasing portion 15 having an outer diameter decreasing toward the distal end 1B of the tip 1, or include the at least one diameter increasing portion 13 and the at least one diameter decreasing portion 15. A most proximal diameter decreasing portion 16 most proximally positioned in the at least one diameter decreasing portion 15 is preferably positioned distally to a most distal diameter increasing portion 14 most distally positioned in the at least one diameter increasing portion 13. Detailed description is made hereinafter to respective configurations.

Preferably, the first region 11 includes a lumen 11L extending from a proximal end 11A to a distal end 11B, and the second region 12 includes the lumen 12L extending from a proximal end 12A to a distal end 12B and communicating with the lumen 11L. This configuration enables insertion of a long body such as a guide wire into the lumens 11L and 12L.

The first region 11 is preferably larger in axial length than the second region 12. This facilitates checking the distal end 4B of the medical tubular body 4. The axial length of the first region 11 corresponds to an axial length from the proximal end 11A to the distal end 11B of the first region 11, and the axial length of the second region 12 corresponds to an axial length from the proximal end 12A to the distal end 12B of the second region 12. The axial length of the first region 11 is preferably 4 mm or more and 20 mm or less, is more preferably 6 mm or more and 15 mm or less, and is further preferably 8 mm or more and 11 mm or less. The axial length of the second region 12 is preferably 0.2 mm or more and 20 mm or less, is more preferably 1.0 mm or more and 15 mm or less, and is further preferably 2.0 mm or more and 10 mm or less.

The axial length of the second region 12 is preferably equal to or more than the maximal radial length of the second region 12, and is more preferably larger than the maximal radial length of the second region 12. This facilitates checking the second region 12.

The diameter increasing portion 13 is preferred to be continuously increased in outer diameter from the proximal end 1A to the distal end 1B in an axial direction 1X of the tip 1. The diameter increasing portion 13 is thus thick on a distal side for improved durability of the tip 1. In addition to such improved durability of the tip 1, the tip 1 is less likely to be caught by the medical tubular body 4 upon removal of the medical tubular body delivery device 10 after the medical tubular body 4 is indwelt, and can have decreased unevenness from an outer tube 3. As depicted in FIGS. 2, 7, and the like, the diameter increasing portion 13 may have an outer surface in a straight shape, a curved shape, or a straight and curved shape in a section in the axial direction.

The diameter decreasing portion 15 is preferred to be continuously decreased in outer diameter from the proximal end IA to the distal end 1B in the axial direction 1X of the tip 1. This facilitates insertion of the tip 1 into a body cavity. As depicted in FIGS. 2, 7, and the like, the diameter decreasing portion 15 may have an outer surface in a straight shape, a curved shape, or a straight and curved shape in a section in an axial direction X.

As depicted in FIGS. 2, 3, and the like, the tip 1 more preferably includes the at least one diameter increasing portion 13 and the at least one diameter decreasing portion 15. This enables exhibition of the effects achieved by the diameter increasing portion 13 and the diameter decreasing portion 15.

As depicted in FIGS. 2, 3, and the like, the tip 1 further preferably includes a constant diameter portion 17 having a constant outer diameter and disposed between the diameter increasing portion 13 and the diameter decreasing portion 15. This leads to higher strength of the tip 1 that can thus easily be pressed into a stenosed portion.

In a case where the tip 1 includes a plurality of diameter increasing portions 13, the most distal diameter increasing portion 14 corresponds to the diameter increasing portion 13 positioned most distally. In another case where the tip 1 includes a single diameter increasing portion 13, the most distal diameter increasing portion 14 corresponds to the single diameter increasing portion 13. Meanwhile, in a case where the tip 1 includes a plurality of diameter decreasing portions 15, the most proximal diameter decreasing portion 16 corresponds to the diameter decreasing portion 15 positioned most proximally. In another case where the tip 1 includes a single diameter decreasing portion 15, the most proximal diameter decreasing portion 16 corresponds to the single diameter decreasing portion 15.

As depicted in FIGS. 6 and 7, the most distal diameter increasing portion 14 and the most proximal diameter decreasing portion 16 may be disposed adjacent to each other. The tip 1 can thus be improved in passableness in a stenosed portion. As depicted in FIGS. 2, 3, and the like, the constant diameter portion 17 may be disposed between the most distal diameter increasing portion 14 and the most proximal diameter decreasing portion 16. This achieves improved strength of the tip 1.

As depicted in FIG. 2 and the like, the most distal diameter increasing portion 14 is preferably smaller in slant angle from the axial direction 1X of the tip 1 than the most proximal diameter decreasing portion 16. The tip 1 is thus less likely to be caught by the medical tubular body 4 upon removal of the medical tubular body delivery device 10 after the medical tubular body 4 is indwelt.

As depicted in FIGS. 2, 3, and the like, the distal end 11B of the first region 11 is preferably positioned distally to a distal end 14B of the most distal diameter increasing portion 14. In the medical tubular body delivery device 10, the distal end 4B of the medical tubular body 4 is typically positioned proximally to the distal end 14B of the most distal diameter increasing portion 14 of the tip 1. Accordingly, positioning the distal end 11B of the first region 11 distally to the distal end 14B of the most distal diameter increasing portion 14 facilitates checking the position of the distal end 4B of the medical tubular body 4.

As depicted in FIGS. 2, 3, and the like, the distal end 11B of the first region 11 is preferably positioned proximally to a proximal end 16A of the most proximal diameter decreasing portion 16. The second region 12 can thus be increased in size. Though not depicted, in a mode in which the tip 1 extends distally to the most proximal diameter decreasing portion 16 as depicted in FIG. 3, the distal end 11B of the first region 11 may be positioned distally to a distal end of the most proximal diameter decreasing portion 16.

As depicted in FIG. 2 and the like, the proximal end 12A of the second region 12 is preferably positioned proximally to the proximal end 16A of the most proximal diameter decreasing portion 16. This facilitates finding a position of the most proximal diameter decreasing portion 16 and finely adjusting a travel direction of the tip 1. In detail, this disposition facilitates delicate operation such as passing the tip 1 through a mesh of a stent already developed at a furcation of a digestive organ, a blood vessel, or the like and conveying the stent to a different stenosed portion.

As depicted in FIGS. 4 and 6, the tip 1 may include the at least one diameter increasing portion 13 and the at least one diameter decreasing portion 15, and the most distal diameter increasing portion 14 may be larger in slant angle from the axial direction 1X of the tip 1 than the most proximal diameter decreasing portion 16. This improves strength of the most distal diameter increasing portion 14 and facilitates transferring pressing force to the tip 1 via an inner tube 2 or the like.

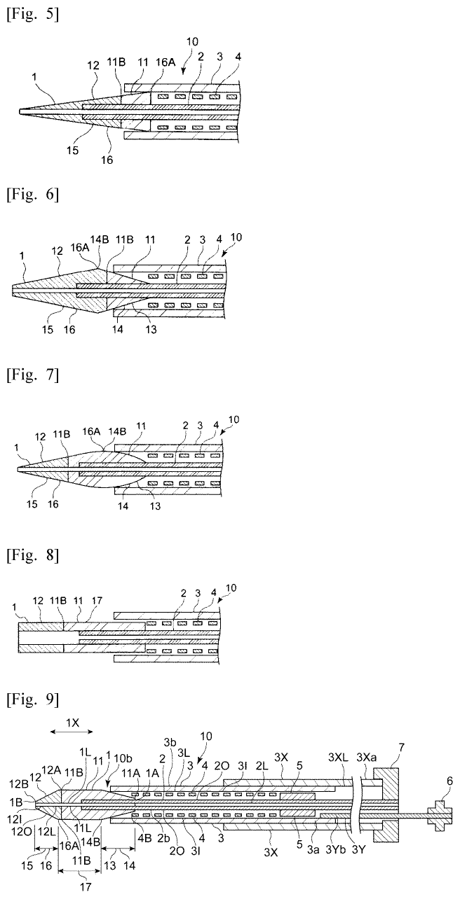

As depicted in FIG. 5, the tip 1 may include the diameter decreasing portion 15 and may not include the diameter increasing portion 13. Alternatively, the tip 1 may include the diameter decreasing portion 15 and may not include the diameter increasing portion 13 and the constant diameter portion 17. The tip 1 can thus be improved in passableness in a stenosed portion.

As depicted in FIGS. 6 and 7, the tip 1 may include the diameter increasing portion 13 and the diameter decreasing portion 15 and may not include the constant diameter portion 17. The tip 1 can thus be improved in passableness in a stenosed portion. Furthermore, in this case, the distal end 11B of the first region 11 may be positioned proximally to the distal end 14B of the most distal diameter increasing portion 14, or may be positioned at the distal end 14B. Alternatively, in this case, the distal end 11B of the first region 11 may be positioned distally to the distal end 14B of the most distal diameter increasing portion 14.

As depicted in FIG. 8, the tip 1 may include the constant diameter portion 17 and may not include the diameter increasing portion 13 and the diameter decreasing portion 15. This enables facilitated manufacture of the tip 1 and a manufacture cost reduction.

The tip 1 can be manufactured through injection molding or extrusion molding of a resin or the like. The tip 1 is preferably disposed at a distal end of a catheter for insertion into a body cavity. The tip 1 is more preferably disposed at a tip end of a medical device configured to deliver a medical tubular body such as a stent into a body cavity. The tip 1 may be disposed at a distal end of a medical device configured to deliver a medical instrument such as a stent having a shape other than a tubular shape, a stent graft having a shape other than a tubular shape, or an obturator having a shape other than a tubular shape. Examples of the shape other than a tubular shape include a spherical shape. Any of these medical devices are preferably adopted along with an endoscope. The tip 1 has been described above. The following description is made to the medical tubular body delivery device 10 according to the embodiment.

As depicted in FIGS. 1 and 2, the medical tubular body delivery device 10 according to the embodiment is configured to deliver the medical tubular body 4 into a living body, and includes the inner tube 2, the outer tube 3 including a lumen 3L where at least part of the inner tube 2 is disposed, the tip 1 being fixed to at least part of a distal end portion 2b of the inner tube 2, and the medical tubular body 4 disposed between an outer surface 2O of the distal end portion 2b of the inner tube 2 and an inner surface 31 of the outer tube 3.

The inner tube 2 includes a lumen 2L configured to receive a guide wire. The inner tube 2 or the like can thus be moved along the guide wire to convey the medical tubular body 4 or the like to a stenosed portion.

At least part of the distal end portion 2b of the inner tube 2 is preferably fixed to an inner surface surrounding a lumen 1L of the tip 1. Accordingly, part of the lumen 1L of the tip 1 preferably has a diameter equal to or more than an outer diameter of the inner tube 2 so as to accommodate the distal end portion 2b of the inner tube 2. Furthermore, the remaining part of the lumen 1L of the tip 1 preferably has a diameter equal to or less than a diameter of the lumen 2L of the inner tube 2. The tip 1 and the inner tube 2 may be fixed directly to each other by thermal welding or the like, or may be fixed indirectly by an adhesive or the like. Furthermore, the inner tube 2 has a distal end preferably positioned proximally to the distal end 11B of the first region 11. This achieves an increase in volume of the second region 12 and facilitates checking the second region 12.

The inner tube 2 has only to be at least partially disposed in the lumen 3L of the outer tube 3. The distal end of the inner tube 2 is preferred to be positioned distally to a distal end of the outer tube 3. This achieves prevention of kinking and a decrease in rigidity unevenness. The inner tube 2 includes a proximal portion that is preferred to be detachably fixed directly or indirectly to a proximal portion of the outer tube 3. This configuration can keep such fixing to prevent unintended development of the medical tubular body 4 during delivery, and can cancel the fixing and move the outer tube 3 to develop the medical tubular body 4 upon indwelling the medical tubular body 4. Such attaching and detaching can be conducted via an outer handle member 7 to be described later.

In a case where the medical tubular body 4 is of a self-expanding type to be described later, the medical tubular body 4 can be kept in a contracted state when being disposed between the outer surface 2O of the distal end portion 2b of the inner tube 2 and the inner surface 31 of the outer tube 3, and can be expanded when moving distally to the outer tube 3. Furthermore, the tip 1 can avoid a malfunction such as development of the medical tubular body 4 distally to the outer tube 3 before the medical tubular body 4 is disposed in a stenosed portion.

As depicted in FIG. 2, the inner tube 2 has a proximal end portion 2a preferably including an inner handle member 6. The outer tube 3 has a proximal end portion 3a preferably including the outer handle member 7. Accordingly, the medical tubular body 4 can be positioned distally to the outer tube 3 to be expanded by pulling the outer tube 3 via the outer handle member 7 in an exemplary state where the inner handle member 6 is kept gripped.

The inner tube 2 is preferably provided with a pusher member 5 fixed to the outer surface 2O. The pusher member 5 facilitates pushing out the medical tubular body 4. The pusher member 5 is preferably a tubular body.

As depicted in FIG. 9, the outer tube 3 may be unfixed to the outer handle member 7 so as to be axially movable. For example, the medical tubular body delivery device 10 preferably includes an outermost tube 3X having a lumen 3XL where at least part of the outer tube 3 is disposed, and a linear member 3Y having a distal end portion 3Yb fixed to the proximal end portion 3a of the outer tube 3. Furthermore, the outermost tube 3X has a proximal end portion 3Xa more preferably including the outer handle member 7. In such a mode, the outer tube 3 can be pulled back to a nearby position in the outermost tube 3X by pulling the linear member 3Y to a nearby position in an exemplary state where the outer handle member 7 is kept gripped, for development of the medical tubular body 4. Examples of the linear member 3Y may include a rotor such as a thumbwheel and an object to be wound around the rotor.

As depicted in FIG. 2, the outer handle member 7 may include a flushable lumen 8. This configuration enables injection into the lumen 3L of the outer tube 3 of a desired liquid from the flushable lumen 8.

The medical tubular body 4 is preferred to include a resin, include a metal, or include the resin and the metal, more preferably includes the metal, and further preferably consists of the metal. The medical tubular body 4 may include a radiopaque substance. The medical tubular body 4 is preferably a stent, a stent graft, an obturator, an injection catheter, or a prosthetic valve, and is more preferably the stent. Examples of the stent include a coiled stent formed by a linear body made of a resin, a metal, or the resin and the metal, a stent laser cut out of a tube and processed, a stent laser cut out of a sheet and then wound into a cylindrical shape and laser welded, a stent obtained by laser welding a linear body and assembling the linear body thus welded, and a stent fabricated by weaving a plurality of linear resins, a plurality of linear metals, or the plurality of linear resins and the plurality of linear metals. The stent is preferably a balloon expanded stent configured to be expanded with a balloon mounted with the stent or a self-expanding stent configured to expand by itself upon removal of an external member inhibiting expansion of the stent, and is more preferably the self-expanding stent.

As depicted in FIG. 2, the distal end 4B of the medical tubular body 4 is preferably positioned proximally to the distal end 11B of the first region 11 of the tip 1. This facilitates finding the position of the distal end 4B of the medical tubular body 4.

The distal end 4B of the medical tubular body 4 is preferably positioned distally to the proximal end 11A of the first region 11 of the tip 1. This inhibits axial movement of the medical tubular body 4 for prevention of positional displacement. Overlapping a distal end portion of the medical tubular body 4 with the tip 1 achieves a decrease in rigidity unevenness and facilitated prevention of kinking.

As depicted in FIG. 10, the outer tube 3 preferably incudes a region 3R having lower total light transmittance measured with the D65 light source in comparison with the first region. This configuration enables finding a position of a distal end portion 3b of the outer tube 3. If the position of the distal end portion 3b of the outer tube 3 can be found, the outer tube 3 can be easily pulled proximally so as to distally release the medical tubular body 4.

As depicted in FIG. 10, the region 3R is preferably positioned proximally to the distal end 11B of the first region 11 of the tip 1. This facilitates checking the region 3R.

The region 3R is preferably positioned distally to the proximal end 11A of the first region 11 of the tip 1. This facilitates checking the region 3R. Preferably, the region 3R includes a colorant, includes a crystalline resin, or includes the colorant and the crystalline resin. This facilitates a decrease in total light transmittance of the region 3R.

Each of the inner tube 2, the outer tube 3, the pusher member 5, and the outermost tube 3X preferably includes a resin. Preferred examples of the resin include polyethylene, polyimide, polyether block amide, polyamide, polyurethane, polyester, silicone, a polyamide-based elastomer, a fluororesin such as PTFE or PFA, and a mixture of any of these resins. The inner tube 2 and the outermost tube 3X may each include a reinforcing member in a tubular shape. Examples of the reinforcing member include a spiral wire rod and a braided wire rod. Examples of the wire rod include stainless steel, titanium, a cobalt-chromium alloy, Ni—Ti, tungsten, polyarylate fiber, aramid fiber, ultrahigh molecular weight polyethylene fiber, PBO fiber, carbon fiber, and a mixture of any of these wire rods. The linear member 3Y is made of a material, examples of which include stainless steel, titanium, a cobalt-chromium alloy, Ni—Ti, and tungsten.

The tip according to the second embodiment of the present invention is configured to be disposed at a distal end portion of a medical device for insertion into a living body, and the tip includes a first region including a proximal end of the tip, and a second region positioned distally to the first region and including a resin and a radiopaque filler. The first region includes a resin and does not include any radiopaque filler, or includes a resin and a radiopaque filler smaller in mass percent content than the radiopaque filler in the second region.

As described above, the tip including the radiopaque filler facilitates finding a position of the tip during X-ray fluoroscopy. Furthermore, the first region of the tip not including any radiopaque filler or including a radiopaque filler smaller in content than the radiopaque filler in the second region achieves relatively improved radiotransparency at the proximal end portion of the tip. Applying the tip thus configured as a tip of a medical tubular body delivery device facilitates accurately finding a position of a distal end or the like of a radiopaque medical tubular body positioned near the proximal end of the tip during X-ray fluoroscopy. The radiopaque filler adopted as a radiopaque substance in the tip is less likely to cause large unevenness in a lumen of the tip in comparison with a case where a radiopaque marker band is provided around the lumen of the tip. This facilitates avoidance of damage to the lumen of the tip due to insertion, removal, or the like of a guide wire.

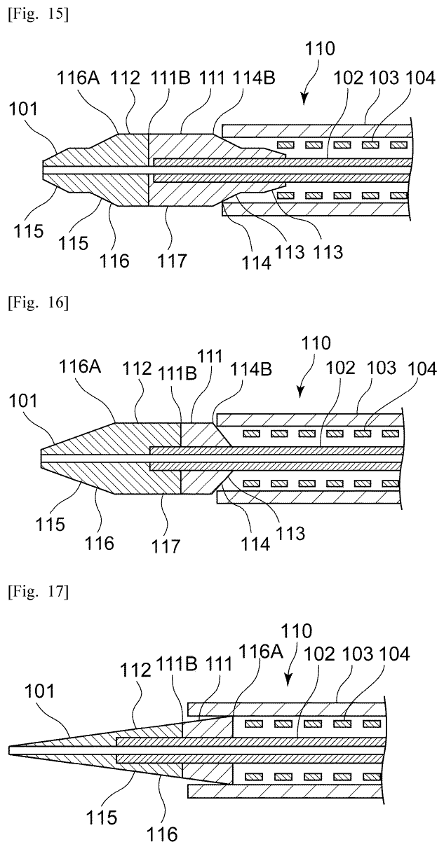

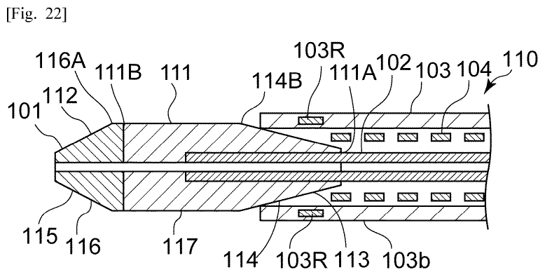

With reference to FIGS. 11 to 22, the following description initially refers to a medical tip according to the embodiment, and then refers to a medical tubular body delivery device according to the embodiment. FIG. 11 is a side view of the medical tubular body delivery device according to the embodiment. FIG. 12 is a sectional view taken in an axial direction indicated in FIG. 11. FIG. 13 is a schematic view exemplifying a tip and a medical tubular body during X-ray fluoroscopy. FIG. 14 is a schematic view of the tip and a medical tubular body according to the embodiment during X-ray fluoroscopy. Each of FIGS. 15 to 20 is a sectional view of a tip depicted in FIG. 12 according to a modification example. FIG. 21 is a sectional view taken in an axial direction of a medical tubular body delivery device according to another embodiment. FIG. 22 is a sectional view of the tip and an outer tube depicted in FIG. 12 according to a modification example.

FIGS. 11 and 12 depict a tip 101 configured to be disposed at a distal end portion of a medical device for insertion into a living body. Specifically, the tip 101 is preferred to be disposed at a distal end portion 110b of a medical tubular body delivery device 110 configured to deliver a medical tubular body 104 into a living body. The tip 101 includes a first region 111 including a proximal end 101A of the tip 101, and a second region 112 positioned distally to the first region 111 and including a resin and a radiopaque filler. Furthermore, the first region 111 includes a resin and does not include any radiopaque filler, or includes a resin and a radiopaque filler smaller in mass percent content than the radiopaque filler in the second region 112.

That is, the tip 101 includes the radiopaque filler in at least the second region 112, or in at least the second region 112 and the first region 111. The tip 101 including the radiopaque filler in a region exhibiting radiopacity is less likely to have large unevenness in a lumen 101L of the tip 101 in comparison with a case where the tip includes a radiopaque marker band. This facilitates avoidance of damage to the lumen 101L of the tip 101 due to insertion, removal, or the like of a guide wire. The tip 101 including the radiopaque filler facilitates finding a position of the tip 101 during X-ray fluoroscopy. Furthermore, the second region 112 is positioned distally to the first region 111 and includes the radiopaque filler. As to be described later, the second region 112 is less likely to be overlapped with the medical tubular body 104 having radiopacity during X-ray fluoroscopy and can be easily checked, and is thus suitable for finding a position of a distal end portion of the tip 101 in a living body.

Furthermore, as described above, the first region 111 of the tip 101 not including any radiopaque filler or including a radiopaque filler smaller in content in comparison with the second region 112 achieves relatively improved radiotransparency at a proximal end portion of the tip 101. This facilitates accurately finding a position of a distal end 104B or the like of the medical tubular body 104 having radiopacity and positioned near the proximal end 101A of the tip 101 during X-ray fluoroscopy. In detail, as to a tip including a radiopaque filler exemplarily at an entirely even concentration during X-ray fluoroscopy, the tip includes a radiopaque region 121 visually recognized as being overlapped with a radiopaque region 120 of the medical tubular body as depicted in FIG. 13. In this case, it is difficult to check a position of a distal end 120B of the radiopaque region 120 of the medical tubular body. In contrast, the first region 111 of the tip 101 according to the embodiment does not include any radiopaque filler or includes a radiopaque filler smaller in content in comparison with the first region 112. This leads to higher visibility of the distal end 120B in the radiopaque region 120 of the medical tubular body 104 as depicted in FIG. 14, and thus enables accurately finding the position of the distal end 104B of the medical tubular body 104. Furthermore, in a case where an outer tube 103 includes a distal end portion 103b provided with a marker band as depicted in FIG. 22 to be referred to later, the first region 111 of the tip 101 according to the embodiment achieves higher visibility of a radiopaque region in the marker band and enables accurately finding a position of the distal end portion 103b or the like of the outer tube 103. Accordingly, the first region 111 of the tip 101 preferably includes no radiopaque filler.

Preferably, the first region 111 includes a resin and does not include any radiopaque substance. The first region 111 is thus almost invisible during X-ray fluoroscopy, to facilitate checking the distal end 104B or the like of the medical tubular body 104. Examples of the radiopaque substance include gold, platinum, silver, tungsten, tantalum, iridium, palladium, rhenium, rhodium, tin, nickel, titanium, an alloy of any of these metals, barium sulfate, a bismuth compound, a tungsten compound, and a mixture of any of these substances. Examples of the alloy include a gold-palladium alloy, a platinum-iridium alloy, an NiTiPd alloy, an NiTiAu alloy, an NiTi alloy, and a mixture of any of these alloys. Examples of forms of the radiopaque substance include a radiopaque filler and a radiopaque marker band.

The first region 111 preferably includes at least one radiotransparent tubular layer containing a resin and not containing any radiopaque substance, and more preferably consists of by the at least one radiotransparent tubular layer. The first region 111 including a radiopaque filler is preferred to include the radiopaque filler at an entirely even concentration.

When the first region 111 includes the radiopaque filler, the radiopaque filler in the first region 111 has a mass percent content that is preferably 0.8 times or less the mass percent content of the radiopaque filler in the second region 112, more preferably 0.5 times or less, further preferably 0.2 times or less, and still further preferably 0.1 times or less. Meanwhile, such a magnification may be 0.01 times or more. Furthermore, the first region 111 including the radiopaque filler is preferred to include a kneaded product containing a resin and the radiopaque filler. Measurement of a content of the radiopaque filler in each region can be achieved by radially cutting each region and obtaining a mass ratio (mass percent) of the radiopaque filler to 100 mass percent of a cut piece. As to an axial length of each region, reference can be made to later description of an axial length of the first region 111 and an axial length of the second region 112. Examples of the radiopaque filler in the first region 111 may include fillers to be exemplified in later description of the radiopaque filler in the second region 112. The radiopaque filler included in the first region 111 may be the same as or different from the radiopaque filler included in the second region 112. As to details of the radiopaque filler included in the first region 111, reference can be made to later description of the radiopaque filler included in the second region 112.

Preferably, the first region 111 includes a lumen 111L extending from a proximal end 111A to a distal end 111B, and the second region 112 includes a lumen 112L extending from a proximal end 112A to a distal end 112B and communicating with the lumen 111L. This configuration enables insertion of a long body such as a guide wire into the lumens 111L and 112L.

The first region 111 is preferably larger in axial length than the second region 112. This facilitates checking the distal end 104B of the medical tubular body 104. The axial length of the first region 111 corresponds to an axial length from the proximal end 111A to the distal end 111B of the first region 111, and the axial length of the second region 112 corresponds to an axial length from the proximal end 112A to the distal end 112B of the second region 112. The axial length of the first region III is preferably 4 mm or more and 20 mm or less, is more preferably 6 mm or more and 15 mm or less, and is further preferably 8 mm or more and 11 mm or less. The axial length of the second region 112 is preferably 0.2 mm or more and 20 mm or less, is more preferably 1.0 mm or more and 15 mm or less, and is further preferably 2.0 mm or more and 10 mm or less.

As described above, the second region 112 includes the resin and the radiopaque filler. The second region 112 including the radiopaque filler facilitates checking the position of the tip 101 during X-ray fluoroscopy. The second region 112 may be positioned proximally to a distal end 101B of the tip 101, but preferably includes the distal end 101B of the tip 101. This facilitates finding a position of the distal end 101B of the tip 101 during X-ray fluoroscopy. Furthermore, this disposition facilitates delicate operation such as passing the tip 101 through a mesh of a stent already developed at a furcation of a digestive organ, a blood vessel, or the like and conveying the stent to a different stenosed portion.

The second region 112 is preferably disposed adjacent to the first region 111. The second region 112 disposed adjacent to the first region 111 leads to more obvious radiopacity of the second region 112 during X-ray fluoroscopy for higher visibility of the second region 112.

The axial length of the second region 112 is preferably equal to or more than the maximal radial length of the second region 112, and is more preferably larger than the maximal radial length of the second region 112. This facilitates checking the second region 112.

The second region 112 preferably includes a kneaded product containing the resin and the radiopaque filler, and preferably consists of the kneaded product. This prevents large unevenness in the lumen 101L of the tip 101 and can provide the tip 101 with radiopacity.

At least part of an inner surface 1121 around the lumen 112L in the second region 112 preferably includes the radiopaque filler. This achieves improved radiopacity at and around a central axis of the second region 112 during X-ray fluoroscopy, and facilitates finding the position of the tip 101. More preferably, the inner surface 1121 of the second region 112 entirely includes the radiopaque filler.

At least part of an outer surface 112O of the second region 112 preferably includes the radiopaque filler. This leads to improved radiopacity at and near an outline of the second region 112 during X-ray fluoroscopy, and facilitates finding the position of the tip 101. More preferably, the outer surface 112O of the second region 112 entirely includes the radiopaque filler.

The second region 112 preferably includes at least one radiopaque tubular layer containing a resin and a radiopaque filler, and more preferably consists of the at least one radiopaque tubular layer. The second region 112 may further include at least one radiotransparent tubular layer stacked in a radial direction of the radiopaque tubular layer and not containing any radiopaque substance.

The second region 112 is preferred to include no radiopaque marker band. This prevents large unevenness in the lumen 101L of the tip 101. Examples of the radiopaque marker band include a marker band made of a radiopaque substance.

The radiopaque filler included in the second region 112 is preferably made of a radiopaque substance. Preferred examples of the radiopaque substance include gold, platinum, silver, tungsten, tantalum, iridium, palladium, rhenium, rhodium, tin, nickel, titanium, an alloy of any of these metals, barium sulfate, a bismuth compound, a tungsten compound, and a mixture of any of these substances. Preferred examples of the alloy include a gold-palladium alloy, a platinum-iridium alloy, an NiTiPd alloy, an NiTiAu alloy, an NiTi alloy, and a mixture of any of these alloys. Examples of forms of the radiopaque filler include radiopaque particles.

The radiopaque filler has an average particle diameter that is preferably from 0.01 μm to 200 μm and is more preferably from 0.1 μm to 50 μm. This achieves exhibition of radiopacity as well as keeping durability of the tip 101. The particle diameter corresponds to the maximum value of a direct distance between two points on an outline of a particle. The average particle diameter can be obtained by calculating an average value of particle diameters of particles within a visual field exemplarily including ten or more visible particles with a scanning electron microscope (SEM), a transmission electron microscope (TEM), or the like.

Preferred examples of a resin provided in the first region 111 or the second region 112 include polyethylene, polypropylene, polyamide, polyurethane, polyester, silicone, a polyamide-based elastomer, a fluororesin such as PTFE or PFA, and a mixture of any of these resins. The resin included in the first region 111 and the resin included the second region 112 may be different from each other, but are preferably the same. The first region 111 and the second region 112 may include an additive such as a hydrolysis inhibitor, a flame retarder, a filler, or a crosslinker.

The distal end 101B of the tip 101 preferably includes a radiopaque filler. This facilitates finding the position of the distal end 101B of the tip 101 during X-ray fluoroscopy. This also facilitates delicate operation such as passing the tip 101 through a mesh of a stent already conveyed into a body cavity and developed.

The tip 101 is preferred to include no radiopaque marker band. This prevents unevenness in the lumen 101L of the tip 101. Examples of the radiopaque marker band include a marker band made of a radiopaque substance.

The tip 101 is preferred to include at least one diameter increasing portion 113 having an outer diameter increasing toward the distal end 101B of the tip 101, include at least one diameter decreasing portion 115 having an outer diameter decreasing toward the distal end 101B of the tip 101, or include the at least one diameter increasing portion 113 and the at least one diameter decreasing portion 115. A most proximal diameter decreasing portion 116 most proximally positioned in the at least one diameter decreasing portion 115 is preferably positioned distally to a most distal diameter increasing portion 114 most distally positioned in the at least one diameter increasing portion 113. Detailed description is made hereinafter to respective configurations.

The diameter increasing portion 113 is preferred to be continuously increased in outer diameter from the proximal end 101A to the distal end 101B in an axial direction 101X of the tip 101. The diameter increasing portion 113 is thus thick on a distal side for improved durability of the tip 101. In addition to such improved durability of the tip 101, the tip 101 is less likely to be caught by the medical tubular body 104 upon removal of the medical tubular body delivery device 110 after the medical tubular body 104 is indwelt, and can have decreased unevenness from the outer tube 103. As depicted in FIGS. 12, 19, and the like, the diameter increasing portion 113 may have an outer surface in a straight shape, a curved shape, or a straight and curved shape in a section in the axial direction.

The diameter decreasing portion 115 is preferred to be continuously decreased in outer diameter from the proximal end 101A to the distal end 101B in the axial direction 101X of the tip 101. This facilitates insertion of the tip 101 into a body cavity. As depicted in FIGS. 12, 19, and the like, the diameter decreasing portion 115 may have an outer surface in a straight shape, a curved shape, or a straight and curved shape in a section in the axial direction X.

As depicted in FIGS. 12, 15, and the like, the tip 101 more preferably includes the at least one diameter increasing portion 113 and the at least one diameter decreasing portion 115. This enables exhibition of the effects achieved by the diameter increasing portion 113 and the diameter decreasing portion 115.

As depicted in FIGS. 12, 15, and the like, the tip 101 further preferably includes a constant diameter portion 117 having a constant outer diameter and disposed between the diameter increasing portion 113 and the diameter decreasing portion 115. This leads to higher strength of the tip 101 that can thus easily be pressed into a stenosed portion.

In a case where the tip 101 includes a plurality of diameter increasing portions 113, the most distal diameter increasing portion 114 corresponds to the diameter increasing portion 113 positioned most distally. In another case where the tip 101 includes a single diameter increasing portion 113, the most distal diameter increasing portion 114 corresponds to the single diameter increasing portion 113. Meanwhile, in a case where the tip 101 includes a plurality of diameter decreasing portions 115, the most proximal diameter decreasing portion 116 corresponds to the diameter decreasing portion 115 positioned most proximally. In another case where the tip 101 includes a single diameter decreasing portion 115, the most proximal diameter decreasing portion 116 corresponds to the single diameter decreasing portion 115.

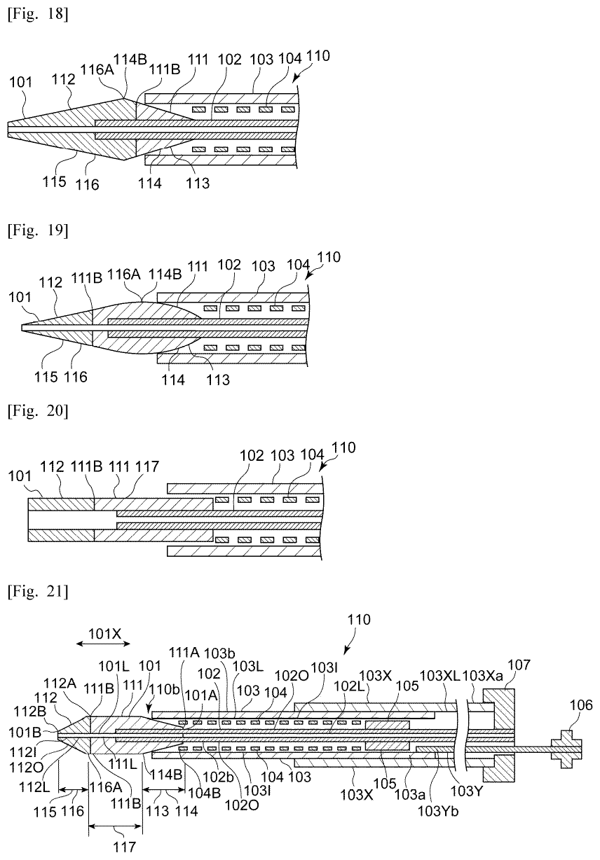

As depicted in FIGS. 18 and 19, the most distal diameter increasing portion 114 and the most proximal diameter decreasing portion 116 may be disposed adjacent to each other. The tip 101 can thus be improved in passableness in a stenosed portion. As depicted in FIGS. 12, 15, and the like, the constant diameter portion 117 may be disposed between the most distal diameter increasing portion 114 and the most proximal diameter decreasing portion 116. This achieves improved strength of the tip 101.

As depicted in FIG. 12 and the like, the most distal diameter increasing portion 114 is preferably smaller in slant angle from the axial direction 101X of the tip 101 than the most proximal diameter decreasing portion 116. The tip 101 is thus less likely to be caught by the medical tubular body 104 upon removal of the medical tubular body delivery device 110 after the medical tubular body 104 is indwelt.

As depicted in FIGS. 12, 15, and the like, the distal end 111B of the first region 111 is preferably positioned distally to a distal end 114B of the most distal diameter increasing portion 114. In the medical tubular body delivery device 110, the distal end 104B of the medical tubular body 104 is typically positioned proximally to the distal end 114B of the most distal diameter increasing portion 114 of the tip 101. Accordingly, positioning the distal end 111B of the first region 111 distally to the distal end 114B of the most distal diameter increasing portion 114 facilitates checking the position of the distal end 104B of the medical tubular body 104 during X-ray fluoroscopy.

As depicted in FIGS. 12, 15, and the like, the distal end 111B of the first region 111 is preferably positioned proximally to a proximal end 116A of the most proximal diameter decreasing portion 116. The second region 112 can thus be increased in size. Though not depicted, in a mode in which the tip 101 extends distally to the most proximal diameter decreasing portion 116 as depicted in FIG. 15, the distal end 111B of the first region 111 may be positioned distally to a distal end of the most proximal diameter decreasing portion 116.

As depicted in FIG. 12 and the like, the proximal end 112A of the second region 112 is preferably positioned proximally to the proximal end 116A of the most proximal diameter decreasing portion 116. This facilitates finding a position of the most proximal diameter decreasing portion 116 during X-ray fluoroscopy and finely adjusting a travel direction of the tip 101 at a furcation of a body cavity. In detail, this disposition facilitates delicate operation such as passing the tip 101 through a mesh of a stent already developed at a furcation of a digestive organ, a blood vessel, or the like and conveying the stent to a different stenosed portion.

As depicted in FIGS. 16 and 18, the tip 101 may include the at least one diameter increasing portion 113 and the at least one diameter decreasing portion 115, and the most distal diameter increasing portion 114 may be larger in slant angle from the axial direction 101X of the tip 101 than the most proximal diameter decreasing portion 116. This improves strength of the most distal diameter increasing portion 114 and facilitates transferring pressing force to the tip 101 via an inner tube 102 or the like.

As depicted in FIG. 17, the tip 101 may include the diameter decreasing portion 115 and may not include the diameter increasing portion 113. Alternatively, the tip 101 may include the diameter decreasing portion 115 and may not include the diameter increasing portion 113 and the constant diameter portion 117. The tip 101 can thus be improved in passableness in a stenosed portion.

As depicted in FIGS. 18 and 19, the tip 101 may include the diameter increasing portion 113 and the diameter decreasing portion 115 and may not include the constant diameter portion 117. The tip 101 can thus be improved in passableness in a stenosed portion. Furthermore, in this case, the distal end 111B of the first region 111 may be positioned proximally to the distal end 114B of the most distal diameter increasing portion 114, or may be positioned at the distal end 114B. Alternatively, in this case, the distal end 111B of the first region 111 may be positioned distally to the distal end 114B of the most distal diameter increasing portion 114.

As depicted in FIG. 20, the tip 101 may include the constant diameter portion 117 and may not include the diameter increasing portion 113 and the diameter decreasing portion 115. This enables facilitated manufacture of the tip 101 and a manufacture cost reduction.

The tip 101 can be manufactured through injection molding or extrusion molding of a resin or the like. The tip 101 is preferably disposed at a distal end of a catheter for insertion into a body cavity. The tip 101 is more preferably disposed at a distal end of a medical device configured to deliver a medical tubular body such as a stent into a body cavity. The tip 101 may be disposed at a distal end of a medical device configured to deliver a medical instrument such as a stent having a shape other than a tubular shape, a stent graft having a shape other than a tubular shape, or an obturator having a shape other than a tubular shape. Examples of the shape other than a tubular shape include a spherical shape. Any of these medical devices are preferably adopted along with an endoscope. The tip 101 has been described above. The following description is made to the medical tubular body delivery device 110 according to the embodiment.

As depicted in FIGS. 11 and 12, the medical tubular body delivery device 110 according to the embodiment is configured to deliver the medical tubular body 104 into a living body, and includes the inner tube 102, the outer tube 103 including a lumen 103L where at least part of the inner tube 102 is disposed, the tip 101 being fixed to at least part of a distal end portion 102b of the inner tube 102, and the medical tubular body 104 disposed between an outer surface 102O of the distal end portion 102b of the inner tube 102 and an inner surface 103I of the outer tube 103.

The inner tube 102 includes a lumen 102L configured to receive a guide wire. The inner tube 102 or the like can thus be moved along the guide wire to convey the medical tubular body 104 or the like to a stenosed portion.

At least part of the distal end portion 102b of the inner tube 102 is preferably fixed to an inner surface surrounding a lumen 101L of the tip 101. Accordingly, part of the lumen 101L of the tip 101 preferably has a diameter equal to or more than an outer diameter of the inner tube 102 so as to accommodate the distal end portion 102b of the inner tube 102. Furthermore, the remaining part of the lumen 101L of the tip 101 preferably has a diameter equal to or less than a diameter of the lumen 102L of the inner tube 102. The tip 101 and the inner tube 102 may be fixed directly to each other by thermal welding or the like, or may be fixed indirectly by an adhesive or the like. Furthermore, the inner tube 102 has a distal end preferably positioned proximally to the distal end 111B of the first region 111. This achieves an increase in volume of the second region 112 and facilitates checking the second region 112.

The inner tube 102 has only to be at least partially disposed in the lumen 103L of the outer tube 103. The distal end of the inner tube 102 is preferred to be positioned distally to a distal end of the outer tube 103. This achieves prevention of kinking and a decrease in rigidity unevenness. The inner tube 102 includes a proximal portion that is preferred to be detachably fixed directly or indirectly to a proximal portion of the outer tube 103. This configuration can keep such fixing to prevent unintended development of the medical tubular body 104 during delivery, and can cancel the fixing and move the outer tube 103 to develop the medical tubular body 104 upon indwelling the medical tubular body 104. Such attaching and detaching can be conducted via an outer handle member 107 to be described later.

In a case where the medical tubular body 104 is of a self-expanding type to be described later, the medical tubular body 104 can be kept in a contracted state when being disposed between the outer surface 102O of the distal end portion 102b of the inner tube 102 and the inner surface 103I of the outer tube 103, and can be expanded when moving distally to the outer tube 103. Furthermore, the tip 101 can avoid a malfunction such as development of the medical tubular body 104 distally to the outer tube 103 before the medical tubular body 104 is disposed in a stenosed portion.

As depicted in FIG. 12, the inner tube 102 has a proximal end portion 102a preferably including an inner handle member 106. The outer tube 103 has a proximal end portion 103a preferably including the outer handle member 107. Accordingly, the medical tubular body 104 can be positioned distally to the outer tube 103 to be expanded by pulling the outer tube 103 via the outer handle member 107 in an exemplary state where the inner handle member 106 is kept gripped.

The inner tube 102 is preferably provided with a pusher member 105 fixed to the outer surface 102O. The pusher member 105 facilitates pushing out the medical tubular body 104. The pusher member 105 is preferably a tubular body.

As depicted in FIG. 21, the outer tube 103 may be unfixed to an outer handle member 107 so as to be axially movable. For example, the medical tubular body delivery device 110 preferably includes an outermost tube 103X including a lumen 103XL where at least part of the outer tube 103 is disposed, and a linear member 103Y having a distal end portion 103Yb fixed to the proximal end portion 103a of the outer tube 103. Furthermore, the outermost tube 103X has a proximal end portion 103Xa more preferably including the outer handle member 107. In such a mode, the outer tube 103 can be pulled back to a nearby position in the outermost tube 103X by pulling the linear member 103Y to a nearby position in an exemplary state where the outer handle member 107 is kept gripped, for development of the medical tubular body 104. Examples of the linear member 103Y may include a rotor such as a thumbwheel and an object to be wound around the rotor.

As depicted in FIG. 12, the outer handle member 107 may include a flushable lumen 108. This configuration enables injection into a lumen 103L of the outer tube 103 of a desired liquid from the flushable lumen 108.

The medical tubular body 104 includes a radiopaque substance. This facilitates checking a position of the medical tubular body 104 during X-ray fluoroscopy. Examples of the radiopaque substance include a radiopaque substance exemplified in the description of the radiopaque substance or the like in the second region 112. The radiopaque substance included in the medical tubular body 104 may be the same as or different from the radiopaque substance included in the second region 112. As to details of the radiopaque substance included in the medical tubular body 104, reference can be made to the description of the radiopaque substance included in the second region 112. The medical tubular body 104 is preferred to include a resin, include a metal, or include the resin and the metal, more preferably includes the metal, and further preferably consists of the metal. The medical tubular body 104 is preferably a stent, a stent graft, an obturator, an injection catheter, or a prosthetic valve, and is more preferably the stent. Examples of the stent include a coiled stent formed by a linear body made of a resin, a metal, or the resin and the metal, a stent laser cut out of a tube and processed, a stent laser cut out of a sheet and then wound into a cylindrical shape and laser welded, a stent obtained by laser welding a linear body and assembling the linear body thus welded, and a stent fabricated by weaving a plurality of linear resins, a plurality of linear metals, or the plurality of linear resins and the plurality of linear metals. The stent is preferably a balloon expanded stent configured to be expanded with a balloon mounted with the stent or a self-expanding stent configured to expand by itself upon removal of an external member inhibiting expansion of the stent, and is more preferably the self-expanding stent.

As depicted in FIG. 12, the distal end 104B of the medical tubular body 104 is preferably positioned proximally to the distal end 111B of the first region 111 of the tip 101. This facilitates finding the position of the distal end 104B of the medical tubular body 104 during X-ray fluoroscopy.

The distal end 104B of the medical tubular body 104 is preferably positioned distally to the proximal end 111A of the first region 111 of the tip 101. This inhibits axial movement of the medical tubular body 104 for prevention of positional displacement. Overlapping a distal end portion of the medical tubular body 104 with the tip 101 achieves a decrease in rigidity unevenness and facilitated prevention of kinking.

As depicted in FIG. 22, the outer tube 103 preferably has the distal end portion 103b including a radiopaque region 103R having radiopacity. This configuration enables finding the position of the distal end portion 103b of the outer tube 103. Finding the position of the distal end portion 103b of the outer tube 103 facilitates visually recognizing a start of movement of the medical tubular body 104.

As depicted in FIG. 22, the radiopaque region 103R is preferably positioned proximally to the distal end 11IB of the first region 111 of the tip 101. This facilitates checking the radiopaque region 103R during X-ray fluoroscopy.

The radiopaque region 103R is preferably positioned distally to the proximal end 111A of the first region 111 of the tip 101. This facilitates checking the radiopaque region 103R during X-ray fluoroscopy.

The radiopaque region 103R is preferred to include a radiopaque marker, include a resin and a radiopaque filler, or include the radiopaque marker, the resin, and the radiopaque filler, and is more preferred to include the radiopaque marker. The distal end portion 103b of the outer tube 103 is typically in no contact with a guide wire and a radiopaque marker at the distal end portion 103b thus has no damage from the guide wire. Furthermore, the radiopaque marker has high radiopacity. This facilitates finding the position of the distal end portion 103b of the outer tube 103.

Each of the inner tube 102, the outer tube 103, a pusher member 105, and the outermost tube 103X preferably includes a resin. Preferred examples of the resin include polyethylene, polyimide, polyether block amide, polyamide, polyurethane, polyester, silicone, a polyamide-based elastomer, a fluororesin such as PTFE or PFA, and a mixture of any of these resins. The resin may include an additive such as a hydrolysis inhibitor, a flame retarder, a filler, or a crosslinker. The inner tube 102 and the outermost tube 103X may each include a reinforcing member in a tubular shape. Examples of the reinforcing member include a spiral wire rod and a braided wire rod. Examples of the wire rod include stainless steel, titanium, a cobalt-chromium alloy, Ni—Ti, tungsten, polyarylate fiber, aramid fiber, ultrahigh molecular weight polyethylene fiber, PBO fiber, carbon fiber, and a mixture of any of these wire rods. The linear member 103Y is made of a material, examples of which include stainless steel, titanium, a cobalt-chromium alloy, Ni—Ti, and tungsten.

The present application claims benefit of priority based on Japanese Patent application No. 2022-019801 and Japanese Patent application No. 2022-019800 filed on Feb. 10, 2022. The entire contents of the specifications of Japanese Patent application No. 2022-019801 and Japanese Patent application No. 2022-019800 filed on Feb. 10, 2022 are incorporated in the present application for reference.

### DESCRIPTION OF REFERENCE SIGNS

1 tip
1A proximal end of tip
1B distal end of tip
1L lumen of tip
1X axial direction of tip
2 inner tube
2a proximal end portion of inner tube
2b distal end portion of inner tube
2O outer surface of inner tube
2L lumen of inner tube
3 outer tube
3a proximal end portion of outer tube
3b distal end portion of outer tube
3L lumen of outer tube
3I inner surface of outer tube
3R region having low total light transmittance
3X outermost tube
3Xa proximal end portion
3XL lumen
3Y linear member
3Yb distal end portion
4 medical tubular body
4B distal end of medical tubular body
5 pusher member
6 inner handle member
7 outer handle member
8 flushable lumen
10 medical tubular body delivery device
10b distal end portion
11 first region of tip
11A proximal end of first region
11B distal end of first region
11L lumen in first region
12 second region of tip
12A proximal end of second region
12B distal end of second region
12L lumen in second region
12I inner surface
12O outer surface
13 diameter increasing portion of tip
14 most distal diameter increasing portion
14B distal end of most distal diameter increasing portion
15 diameter decreasing portion of tip
16 most proximal diameter decreasing portion
16A proximal end of most proximal diameter decreasing portion
17 constant diameter portion
101 tip
101A proximal end of tip
101B distal end of tip
101L lumen of tip
101X axial direction of tip
102 inner tube
102a proximal end portion of inner tube
102b distal end portion of inner tube
102O outer surface of inner tube
102L lumen of inner tube
103 outer tube
103a proximal end portion of outer tube
103b distal end portion of outer tube
103L lumen of outer tube
103I inner surface of outer tube
103R radiopaque region
103X outermost tube
103Xa proximal end portion
103XL lumen
103Y linear member
103Yb distal end portion
104 medical tubular body
104B distal end of medical tubular body
105 pusher member
106 inner handle member
107 outer handle member
108 flushable lumen
110 medical tubular body delivery device
110b distal end portion
111 first region of tip
111A proximal end of first region
111B distal end of first region
111L lumen in first region
112 second region of tip
112A proximal end of second region
112B distal end of second region
112L lumen in second region
112I inner surface
112O outer surface
113 diameter increasing portion of tip
114 most distal diameter increasing portion
114B distal end of most distal diameter increasing portion
115 diameter decreasing portion of tip
116 most proximal diameter decreasing portion
116A proximal end of most proximal diameter decreasing portion
117 constant diameter portion
120 radiopaque region of medical tubular body
120B distal end
121 radiopaque region of tip

1. A tip configured to be disposed at a distal end portion of a medical device for insertion into a living body, the tip comprising:
a first region including a proximal end of the tip; and
a second region positioned distally to the first region; wherein
the first region has higher total light transmittance measured with a D65 light source in comparison with the second region.

2. The tip according to claim 1, wherein the first region has less haze measured with the D65 light source in comparison with the second region.

3. The tip according to claim 1, wherein
the second region includes a colorant, and
the first region does not include any colorant or includes a colorant smaller in mass percent content than the colorant in the second region.

4. The tip according to claim 3, wherein
the colorant is a pigment, a dye, or a mixture of the pigment and the dye.

5. The tip according to claim 3, wherein
the first region includes a lumen extending from a proximal end to a distal end, and
the second region includes a lumen extending from a proximal end to a distal end and communicating with the lumen in the first region.

6. The tip according to claim 5, wherein at least part of an inner surface of the lumen in the second region includes the colorant.

7. The tip according to claim 3, wherein at least part of an outer surface of the second region includes the colorant.

8. The tip according to claim 3, wherein the tip has a distal end including the colorant.

9. The tip according to claim 1, wherein
the second region includes a crystalline resin, and
the first region does not include any crystalline resin or includes a crystalline resin smaller in mass percent content than the crystalline resin in the second region.

10. The tip according to claim 1, wherein
the second region includes a crystalline resin, and
the first region does not include any crystalline resin or includes a crystalline resin lower in crystallinity than the crystalline resin in the second region.

11. The tip according to claim 1, wherein the first region includes an amorphous resin.

12. The tip according to claim 1, the tip
comprising at least one diameter increasing portion having an outer diameter increasing toward a distal end of the tip, comprising at least one diameter decreasing portion having an outer diameter decreasing toward the distal end of the tip, or comprising the at least one diameter increasing portion and the at least one diameter decreasing portion; wherein
a most proximal diameter decreasing portion most proximally positioned in the at least one diameter decreasing portion is positioned distally to a most distal diameter increasing portion most distally positioned in the at least one diameter increasing portion.

13. The tip according to claim 12, wherein the most distal diameter increasing portion is smaller in slant angle from an axial direction of the tip than the most proximal diameter decreasing portion.

14. The tip according to claim 12, wherein the first region has a distal end positioned distally to a distal end of the most distal diameter increasing portion.

15. The tip according to claim 12, wherein the first region has a distal end positioned proximally to a proximal end of the most proximal diameter decreasing portion.

16. A medical tubular body delivery device configured to deliver a medical tubular body into a living body, the medical tubular body delivery device comprising:
an inner tube;
an outer tube including a lumen, at least part of the inner tube being disposed in the lumen;
the tip according to any one of claims 1 to 15, the tip being fixed to at least part of the distal end portion of the inner tube; and
a medical tubular body disposed between an outer surface of the distal end portion of the inner tube and an inner surface of the outer tube.

17. The medical tubular body delivery device according to claim 16, wherein the medical tubular body has a distal end positioned proximally to a distal end of the first region of the tip.

18. The medical tubular body delivery device according to claim 16, wherein the medical tubular body has a distal end positioned distally to a proximal end of the first region of the tip.

19. A tip configured to be disposed at a distal end portion of a medical device for insertion into a living body, the tip comprising:
a first region including a proximal end of the tip; and
a second region positioned distally to the first region and including a resin and a radiopaque filler, wherein
the first region includes a resin and does not include any radiopaque filler, or includes a resin and a radiopaque filler smaller in mass percent content than the radiopaque filler in the second region.

20. The tip according to claim 19, wherein the first region includes the resin and does not include any radiopaque substance.

21. The tip according to claim 19, wherein
the first region includes a lumen extending from a proximal end to a distal end, and
the second region includes a lumen extending from a proximal end to a distal end and communicating with the lumen in the first region.

22. The tip according to claim 21, wherein at least part of an inner surface of the lumen in the second region includes the radiopaque filler.

23. The tip according to claim 19, wherein at least part of an outer surface of the second region includes the radiopaque filler.

24. The tip according to claim 19, wherein the tip has a distal end including the radiopaque filler.

25. The tip according to claim 19, the tip
comprising at least one diameter increasing portion having an outer diameter increasing toward a distal end of the tip, comprising at least one diameter decreasing portion having an outer diameter decreasing toward the distal end of the tip, or comprising the at least one diameter increasing portion and the at least one diameter decreasing portion; wherein
a most proximal diameter decreasing portion most proximally positioned in the at least one diameter decreasing portion is positioned distally to a most distal diameter increasing portion most distally positioned in the at least one diameter increasing portion.

26. The tip according to claim 25, wherein the most distal diameter increasing portion is smaller in slant angle from an axial direction of the tip than the most proximal diameter decreasing portion.

27. The tip according to claim 25, wherein the first region has a distal end positioned distally to a distal end of the most distal diameter increasing portion.

28. The tip according to claim 25, wherein the first region has a distal end positioned proximally to a proximal end of the most proximal diameter decreasing portion.

29. A medical tubular body delivery device configured to deliver a medical tubular body into a living body, the medical tubular body delivery device comprising:
an inner tube;
an outer tube including a lumen, at least part of the inner tube being disposed in the lumen;
the tip according to any one of claims 19 to 28, the tip being fixed to at least part of a distal end portion of the inner tube; and
a medical tubular body disposed between an outer surface of the distal end portion of the inner tube and an inner surface of the outer tube, and including a radiopaque substance.

30. The medical tubular body delivery device according to claim 29, wherein the medical tubular body has a distal end positioned proximally to a distal end of the first region of the tip.

31. The medical tubular body delivery device according to claim 29, wherein the medical tubular body has a distal end positioned distally to a proximal end of the first region of the tip.

32. The medical tubular body delivery device according to claim 29, wherein the outer tube has a distal end portion including a radiopaque region having radiopacity.

33. The medical tubular body delivery device according to claim 32, wherein the radiopaque region is positioned proximally to a distal end of the first region of the tip.

34. The medical tubular body delivery device according to claim 32, wherein the radiopaque region is positioned distally to a proximal end of the first region of the tip.

35. The medical tubular body delivery device according to claim 32, wherein the radiopaque region includes a radiopaque marker, includes a resin and a radiopaque filler, or includes the radiopaque marker, the resin, and the radiopaque filler.
